(19) United States
(12) Patent Application Publication
(10) Pub. No.: US 2011/0118773 A1
(43) Pub. Date: May 19, 2011

(54) ELLIPTICAL DEVICE FOR TREATING AFTERLOAD

(75) Inventors: Yossi Gross, Moshav Mazor (IL); Amir Dagan, Kibbutz Megiddo (IL); Nitai Hanani, Haifa (IL)

(73) Assignee: RAINBOW MEDICAL LTD., Herzliya (IL)

(21) Appl. No.: 12/959,126

(22) Filed: Dec. 2, 2010

Related U.S. Application Data
(63) Continuation-in-part of application No. 11/995,904, filed on Mar. 11, 2008.
(60) Provisional application No. 60/702,491, filed on Jul. 25, 2005.
(60) Provisional application No. 60/721,728, filed on Sep. 28, 2005.

(51) Int. Cl. A61M 29/00

(52) U.S. Cl. 606/194

(57) ABSTRACT

Apparatus and methods are provided, including an elongate element that is inserted into a subject's artery via a catheter, the catheter being configured to be withdrawn from the artery subsequent to the insertion. Expandable elements are disposed on the elongate element, the expandable elements comprising respective distal tips that are substantially aligned with the elongate element. During insertion of the elongate element via the catheter, the expandable elements are in contracted states thereof. In response to the withdrawal of the catheter from the artery, the expandable elements cause the artery to change a shape thereof, by the expandable elements expanding such that the distal tips of the expandable elements contact respective contact points on the wall of the artery. Subsequently, the distal tips of the expandable elements are freed from the contact points upon pulling of the elongate element. Other embodiments are also described.

### CROSS REFERENCES TO RELATED APPLICATIONS

The present application is a continuation-in-part of U.S. patent application Ser. No. 11/995,904, which is the US National Phase of PCT Application PCT/IL2006/00856, entitled “Electrical stimulation of blood vessels,” to Gross, filed Jul. 25, 2006, which claims the benefit of: (a) U.S. Provisional Application 60/702,491, filed Jul. 25, 2005, entitled, “Electrical stimulation of blood vessels,” and (b) U.S. Provisional Application 60/721,728, filed Sep. 28, 2005, entitled, “Electrical stimulation of blood vessels.” All of the aforementioned references are incorporated herein by reference.

### FIELD OF EMBODIMENTS OF THE INVENTION

Some applications of the present invention generally relate to implantable medical apparatus. Specifically, some applications of the present invention relate to implantable medical apparatus for treating afterload.

### BACKGROUND

Heart failure is a chronic cardiac condition characterized by a deficiency in the ability of the heart to pump blood. Decreased cardiac output to the systemic circulation typically increases venous blood pressure, which often leads to blood backing up in the lungs. Low cardiac output also results in decreased blood perfusion to organs, such as the liver, kidney, brain, and heart itself. Over time, the effects of heart failure contribute to a worsening of the condition. Reduced blood supply to the heart causes less effective contraction of the heart. At the same time, higher venous blood pressure increases the heart preload. To compensate, the heart attempts to increase output by increasing muscle strength, which leads to myocardial hypertrophy (enlargement of the heart with thickening and stiffening of the heart wall). These conditions in turn lead to reduced cardiac output, resulting in a vicious cycle.

Left ventricular afterload is the pressure that the left ventricle has to generate in order to eject blood out of the left ventricle. Counterpulsation is a technique for assisting the circulation by decreasing the afterload of the left ventricle and augmenting the diastolic pressure. Devices for achieving counterpulsation include intra-aortic balloons, pumping devices implantable in the chest, and external devices that apply a negative pressure to the lower extremities during cardiac systole. Counterpulsation devices are typically synchronized with a patient's cardiac cycle to apply pressure to blood vessels of the patient during diastole, and to remove the applied pressure immediately prior to systole, so as to increase stroke volume by decreasing afterload, to reduce heart workload, and to maintain or increase coronary perfusion.

### SUMMARY OF EMBODIMENTS

For some applications of the present invention, a subject is identified as suffering from increased afterload. A self-expandable device is placed inside an artery of the subject, typically, the aorta. The device is configured such that when no pressure is exerted on the device, the device has a tendency to cause the artery to be elliptical in cross-section. During diastole, the device forces the artery into an elliptical shape, such that it reduces the cross-sectional area of the artery, relative to the diastolic cross-sectional area of the aorta when the device is not placed inside the artery. During systole, the blood pressure forces the device to assume a more circular shape.

In the aforementioned manner, the device causes the increase in cross-sectional area of the aorta from diastole to systole to be greater than the increase would be in the absence of the device. Thus, when the device is implanted, there is a larger increase in the volume of the aorta from diastole to systole, to accommodate the blood flow into the aorta during systole, than in the absence of the device. Therefore, it is relatively easier for a large quantity of blood to flow from the heart during systole in the presence of the device, than in the absence of the device, i.e., the device reduces afterload. In effect, the compliance of the post-cardiac vasculature, e.g., the aorta, over the course of the subject's cardiac cycle is increased. For some applications, the device causes increased perfusion of the coronary arteries relative to perfusion of the coronary arteries in the absence of the device.

For some applications, a catheter is inserted into the subject's artery, e.g., the subject's aorta. An elongate element is inserted into the subject's artery via the catheter, a plurality of expandable elements being disposed on the elongate element. During the insertion of the elongate element via the catheter, the expandable elements are in contracted states thereof, being constrained within the catheter. Subsequent to the insertion of the elongate element into the artery, the catheter is withdrawn from the artery. The withdrawal of the catheter from the artery causes the expandable elements to expand against the artery. The expandable elements typically expand in such a manner that they cause the artery to have a more elliptical shape during diastole of the subject than the artery would have in the absence of the expandable elements, as described hereinabove.

For some applications, apparatus and techniques described herein are used in combination with those described in U.S. Pat. No. 7,614,998 to Gross, and/or US 2008/0215117 to Gross, both of which applications are incorporated herein by reference.

There is therefore provided, in accordance with some applications of the present invention, apparatus, including:

a catheter configured to be inserted into an artery of a subject;

an elongate element configured to be inserted into the subject's artery via the catheter, the catheter being configured to be withdrawn from the artery subsequent to the insertion of the elongate element into the artery; and

a plurality of expandable elements disposed on the elongate element, the expandable elements including respective distal tips that are substantially aligned with the elongate element, the expandable elements being configured:
during insertion of the elongate element via the catheter, to be in contracted states thereof,
in response to the withdrawal of the catheter from the artery, to cause the artery to change a shape thereof, by the expandable elements expanding, the expansion of the expandable elements causing the distal tips of the expandable elements to contact respective contact points on the wall of the artery, and
subsequently, for the distal tips of the expandable elements to be freed from their contact points with the wall of the artery upon pulling of the elongate element.

For some applications, the expandable elements are configured to be withdrawn from the artery by pulling the elongate element proximally.

For some applications, the expandable elements are configured to be withdrawn from the artery by advancing a catheter over the elongate element, and, subsequently, removing the catheter from the artery with the elongate element disposed inside the catheter.

For some applications, the expandable elements are configured to cause the artery to change the shape thereof by causing the artery to assume a first cross-sectional shape during a first phase of a cardiac cycle, and a second cross-sectional shape during a second phase of the cardiac cycle.

For some applications, the expandable elements are configured to cause an end-diastolic cross-sectional area of the artery to be 5-30 percent lower than the end-diastolic cross-sectional area of the artery in the absence of the expandable elements, by causing the artery to have the first and second cross-sectional shapes.

For some applications, the expandable elements are configured to cause an end-diastolic cross-sectional area of the artery to be 30-60 percent lower than the end-diastolic cross-sectional area of the artery in the absence of the expandable elements, by causing the artery to have the first and second cross-sectional shapes.

For some applications, the expandable elements are configured to cause an end-diastolic cross-sectional area of the artery to be 60-90 percent lower than the end-diastolic cross-sectional area of the artery in the absence of the expandable elements, by causing the artery to have the first and second cross-sectional shapes.

For some applications, the expandable elements are configured to cause an end-diastolic cross-sectional area of the artery to be more than 90 percent lower than an end-diastolic cross-sectional area of the artery in the absence of the expandable elements, by causing the artery to have the first and second cross-sectional shapes.

For some applications, the expandable elements are configured to cause the artery to have the first and second elliptical cross-sectional shapes, a ratio of (a) a major axis of the artery when assuming the first cross-sectional elliptical shape at end-diastole to (b) a major axis of the artery when assuming the second cross-sectional elliptical shape at end-diastole being between 1.1 and 1.5.

For some applications, the expandable elements are configured to cause the artery to assume the first and second shapes, a cross-sectional area of the artery when the artery assumes the second shape being greater than a cross-sectional area of the artery when the artery assumes the first shape.

For some applications, the expandable elements are configured to cause the artery to assume the first and second cross-sectional shapes, a cross-sectional area of the artery when the artery assumes the first cross-sectional shape being between 10 and 30 percent less than a cross-sectional area of the artery when the artery assumes the second cross-sectional shape.

There is further provided, in accordance with some applications of the present invention, a method, including:

inserting a catheter into an artery of a subject;

inserting an elongate element into the subject's artery via the catheter, a plurality of expandable elements being disposed on the elongate element, and being in contracted states thereof during the insertion of the elongate element via the catheter;

subsequent to the insertion of the elongate element into the artery changing a shape of the artery, by expanding the expandable elements against respective contact points on the artery wall, by withdrawing the catheter; and,

subsequently, freeing the expandable elements from their contact points with the wall of the artery by pulling the elongate element.

For some applications, the method further includes determining a native compliance of the subject's artery, and in response to the determining, selecting elements to be used as the expandable elements.

The present invention will be more fully understood from the following detailed description of embodiments thereof, taken together with the drawings, in which:

### BRIEF DESCRIPTION OF THE DRAWINGS

FIGS. 1A and 1B are schematic illustrations of a self-expandable device inside a subject's artery during, respectively, diastole and systole, in accordance with some applications of the present invention; and

FIG. 2 is a schematic illustration of expandable elements of the device expanding, as a catheter is withdrawn from the subject's artery, in accordance with some applications of the present invention.

### DETAILED DESCRIPTION OF EMBODIMENTS

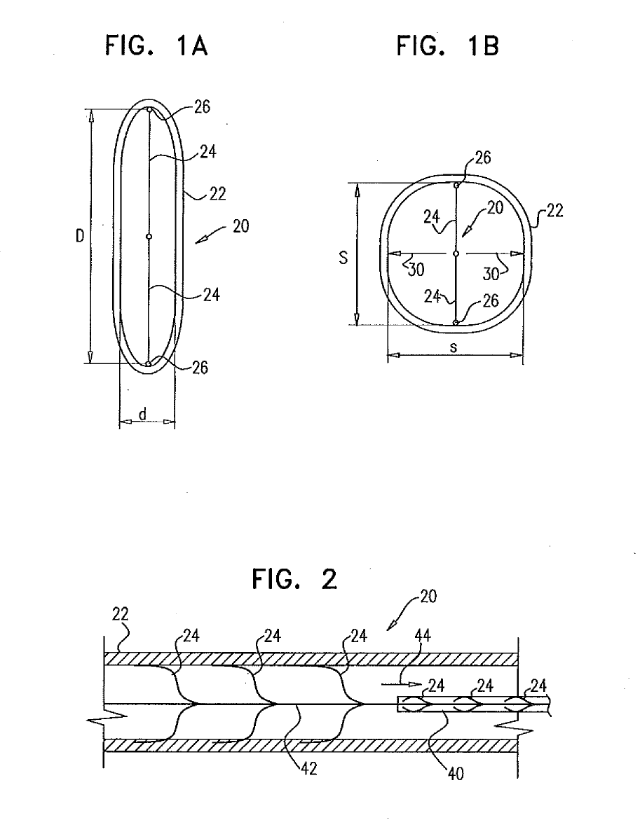

Reference is now made to FIGS. 1A and 1B, which are schematic illustrations of self-expandable device 20 inside a subject's artery 22 during, respectively, diastole and systole, in accordance with some applications of the present invention. For some applications, a subject is identified as suffering from increased afterload. In response, self-expandable device 20 is placed inside artery 22, which is typically the aorta.

Device 20 is configured such that when the device is inside artery 22, expandable elements 24 of the device have a tendency to expand during diastole so as to cause the artery to assume a more elliptical shape than in the absence of the device. During diastole, as shown in FIG. 1A, blood pressure within artery 22 decreases relative to that during systole, so that less blood pressure is applied to the wall of the artery. As a result, less pressure is exerted, by the artery wall, onto blood-vessel-contacting portions 26 of device 20 than during systole. Therefore, expandable elements 24 expand, thereby causing the artery to assume an elliptical shape.

During systole, blood pressure within artery 22 increases, causing the walls of the artery to move in the direction indicated by arrows 30, thereby applying force to expandable elements 24. This causes the artery to assume a more circular cross-section, thereby increasing the cross-sectional area of the artery relative to the cross-sectional area of the artery during diastole, as shown in FIG. 1B. The expandable elements are pushed toward the longitudinal axis of the artery, resulting, at end-systole, in the artery having a more circular cross-sectional shape (i.e., with a smaller difference between lengths of the major axis S and minor axis s of the cross-sectional shape) than during diastole (when the artery has major axis D and minor axis d), as is schematically shown in FIG. 1B. It is noted that for some applications, even at end-systole, the artery does not have a circular cross-section; nevertheless, the artery is more circular than during diastole.

Typically, one or both of the diastolic and systolic cross-sectional shapes are elliptical, and have major axes with different lengths D and S, respectively. For some applications, the systolic cross-sectional shape is substantially circular. For some applications, a ratio of D to S is greater than 1.1, 1.2, 1.3, 1.4, or 1.5. Implantation of device 20 typically results in end-diastolic cross-sectional area of the artery being between less than the end-systolic cross-sectional area of the artery. For example, by way of illustration and not limitation, the end-diastolic cross-sectional area of the artery may be about 10% and about 30% less than end-systolic cross-sectional area of the artery.

The end-diastolic cross-sectional area of the artery in the presence of device 20 is typically substantially lower than the end-diastolic cross-sectional area of the artery in the absence of the device. Depending on the size and spring constant of expandable elements 24, the end-diastolic cross-sectional area of the artery may be 5-30% lower, 30-60% lower, or 60-90% lower than the end-diastolic cross-sectional area of the artery in the absence of treatment. For some applications, suitable spring parameters are chosen such that at end diastole the artery is effectively, momentarily, emptied (e.g., the end-diastolic cross-sectional area of the artery is less than 10% of the end-diastolic cross-sectional area of the artery in the absence of treatment).

For some applications, prior to implantation of device 20 in a patient, the compliance of artery 22 in the patient is assessed. Expandable elements 24 having appropriate expansion characteristics are selected responsive to the assessed compliance. For example, expandable elements having a suitable shape and/or having a suitable spring constant may be selected for implantation responsive to the assessed compliance. For some applications, the compliance of artery 22 is assessed via an invasive diagnostic procedure. Alternatively or additionally, the compliance is measured via a non-invasive diagnostic procedure, e.g., a blood test, an ultrasound assessment, or another test known in the art for assessing blood vessel compliance.

For some applications, artery 22 includes a peripheral artery, such as a peripheral artery having a diameter of at least about 1 cm, such as the femoral artery.

FIG. 2 is a schematic illustration of expandable elements 24 of the device expanding, as a catheter 40 is withdrawn from the subject's artery, in accordance with some applications of the present invention.

For some applications, device 20 is adapted to be inserted into artery 22, e.g., an aorta, using catheter 40. For example, the device is inserted transcatheterally, via a femoral artery of the subject. Expandable elements 24 are stored in the catheter in a contracted position. After the catheter has been advanced to a desired location in the artery, the catheter is withdrawn from the artery, exposing the expandable elements and allowing them to expand against the wall of the artery. FIG. 2 shows catheter 20 being withdrawn in the direction of arrow 44. As shown, the expandable elements that are still inside the catheter are in contracted states thereof. The expandable elements that are not constrained by the catheter have expanded into contact with the artery wall.

For some applications, catheter 40, or a different catheter, is used to remove device 20 from artery 22 after treatment. The catheter is advanced over expandable elements 24, causing the expandable elements to contract and be stored in the catheter. The catheter, holding the expandable elements, is then withdrawn from the artery. Alternatively, the expandable elements are withdrawn without using the catheter, because their orientation within the artery allows them to be freed from their contact points with the artery by pulling elongate element 42, which connects the expandable elements.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus, comprising:
a catheter configured to be inserted into an artery of a subject;
an elongate element configured to be inserted into the subject's artery via the catheter, the catheter being configured to be withdrawn from the artery subsequent to the insertion of the elongate element into the artery; and
a plurality of expandable elements disposed on the elongate element, the expandable elements comprising respective distal tips that are substantially aligned with the elongate element, the expandable elements being configured:
during insertion of the elongate element via the catheter, to be in contracted states thereof,
in response to the withdrawal of the catheter from the artery, to cause the artery to change a shape thereof, by the expandable elements expanding, the expansion of the expandable elements causing the distal tips of the expandable elements to contact respective contact points on the wall of the artery, and
subsequently, for the distal tips of the expandable elements to be freed from their contact points with the wall of the artery upon pulling of the elongate element.

2. The apparatus according to claim 1, wherein the expandable elements are configured to be withdrawn from the artery by pulling the elongate element proximally.

3. The apparatus according to claim 1, wherein the expandable elements are configured to be withdrawn from the artery by advancing a catheter over the elongate element, and, subsequently, removing the catheter from the artery with the elongate element disposed inside the catheter.

4. The apparatus according to claim 1, wherein the expandable elements are configured to cause the artery to change the shape thereof by causing the artery to assume a first cross-sectional shape during a first phase of a cardiac cycle, and a second cross-sectional shape during a second phase of the cardiac cycle.

5. The apparatus according to claim 4, wherein the expandable elements are configured to cause an end-diastolic cross-sectional area of the artery to be 5-30 percent lower than the end-diastolic cross-sectional area of the artery in the absence of the expandable elements, by causing the artery to have the first and second cross-sectional shapes.

6. The apparatus according to claim 4, wherein the expandable elements are configured to cause an end-diastolic cross-sectional area of the artery to be 30-60 percent lower than the end-diastolic cross-sectional area of the artery in the absence of the expandable elements, by causing the artery to have the first and second cross-sectional shapes.

7. The apparatus according to claim 4, wherein the expandable elements are configured to cause an end-diastolic cross-sectional area of the artery to be 60-90 percent lower than the end-diastolic cross-sectional area of the artery in the absence of the expandable elements, by causing the artery to have the first and second cross-sectional shapes.

8. The apparatus according to claim 4, wherein the expandable elements are configured to cause an end-diastolic cross-sectional area of the artery to be more than 90 percent lower than an end-diastolic cross-sectional area of the artery in the absence of the expandable elements, by causing the artery to have the first and second cross-sectional shapes.

9. The apparatus according to claim 4, wherein the expandable elements are configured to cause the artery to have the first and second elliptical cross-sectional shapes, a ratio of (a) a major axis of the artery when assuming the first cross-sectional elliptical shape at end-diastole to (b) a major axis of the artery when assuming the second cross-sectional elliptical shape at end-diastole being between 1.1 and 1.5.

10. The apparatus according to claim 4, wherein the expandable elements are configured to cause the artery to assume the first and second shapes, a cross-sectional area of the artery when the artery assumes the second shape being greater than a cross-sectional area of the artery when the artery assumes the first shape.

11. The apparatus according to claim 10, wherein the expandable elements are configured to cause the artery to assume the first and second cross-sectional shapes, a cross-sectional area of the artery when the artery assumes the first cross-sectional shape being between 10 and 30 percent less than a cross-sectional area of the artery when the artery assumes the second cross-sectional shape.

12. A method, comprising:
inserting a catheter into an artery of a subject;
inserting an elongate element into the subject's artery via the catheter, a plurality of expandable elements being disposed on the elongate element, and being in contracted states thereof during the insertion of the elongate element via the catheter;
subsequent to the insertion of the elongate element into the artery changing a shape of the artery, by expanding the expandable elements against respective contact points on the artery wall, by withdrawing the catheter; and,
subsequently, freeing the expandable elements from their contact points with the wall of the artery by pulling the elongate element.

13. The method according to claim 12, further comprising determining a native compliance of the subject's artery, and in response to the determining, selecting elements to be used as the expandable elements.

14. The method according to claim 12, wherein freeing the expandable elements from their contact points comprises advancing a catheter over the elongate element, and, subsequently, removing the catheter from the artery with the elongate element disposed inside the catheter.

15. The method according to claim 12, wherein changing the shape of the artery comprises causing the artery to assume a first cross-sectional shape during a first phase of a cardiac cycle, and a second cross-sectional shape during a second phase of the cardiac cycle.

16. The method according to claim 15, wherein changing the shape of the artery comprises causing an end-diastolic cross-sectional area of the artery to be 5-30 percent lower than the end-diastolic cross-sectional area of the artery in the absence of the expandable elements.

17. The method according to claim 15, wherein changing the shape of the artery comprises causing an end-diastolic cross-sectional area of the artery to be 30-60 percent lower than the end-diastolic cross-sectional area of the artery in the absence of the expandable elements.

18. The method according to claim 15, wherein changing the shape of the artery comprises causing an end-diastolic cross-sectional area of the artery to be 60-90 percent lower than the end-diastolic cross-sectional area of the artery in the absence of the expandable elements.

19. The method according to claim 15, wherein changing the shape of the artery comprises causing an end-diastolic cross-sectional area of the artery to be more than 90 percent lower than an end-diastolic cross-sectional area of the artery in the absence of the expandable elements.

20. The method according to claim 15, wherein changing the shape of the artery comprises causing the artery to have the first and second elliptical cross-sectional shapes, a ratio of (a) a major axis of the artery when assuming the first cross-sectional elliptical shape at end-diastole to (b) a major axis of the artery when assuming the second cross-sectional elliptical shape at end-diastole being between 1.1 and 1.5.

21. The method according to claim 15, wherein changing the shape of the artery comprises causing the artery to assume the first and second shapes, a cross-sectional area of the artery when the artery assumes the second shape being greater than a cross-sectional area of the artery when the artery assumes the first shape.

22. The method according to claim 21, wherein changing the shape of the artery comprises causing the artery to assume the first and second cross-sectional shapes, a cross-sectional area of the artery when the artery assumes the first cross-sectional shape being between 10 and 30 percent less than a cross-sectional area of the artery when the artery assumes the second cross-sectional shape.
